(19) United States
(12) Patent Application Publication
(10) Pub. No.: US 2007/0029400 A1
(43) Pub. Date: Feb. 8, 2007

(54) Scented pharmaceutical or neutraceutical vessel

(75) Inventors: Ronald J. Magargee, Brandon, MS (US); Genevieve H. Kuhn, Albuquerque, NM (US); Stefan O. Dick, Weichering (DE)

Correspondence Address: Scott R. Cox, Ste. 2100, 500 W. Jefferson St., Louisville, KY, 40202, US

(73) Assignee: Sud-Chemie Inc., Louisville, KY (US)

(21) Appl. No.: 11/525,388

(22) Filed: Sep. 22, 2006

Related U.S. Application Data
(63) Continuation-in-part of application No. 10/996,916, filed on Nov. 24, 2004.
Application No. 10/996,916 is a continuation-in-part of application No. 10/328,579, filed on Dec. 24, 2002.
(60) Provisional application No. 60/720,138, filed on Sep. 23, 2005.
(60) Provisional application No. 60/375,841, filed on Apr. 25, 2002.

(51) Int. Cl. A24F 25/00; A61L 9/04

(52) U.S. Cl. 239/34; 239/54; 239/55

(57) ABSTRACT

A pharmaceutical or neutraceutical vessel containing a scented material which includes a pharmaceutical or neutraceutical container and a scented, shaped material, placed within or formed as part of the pharmaceutical or neutraceutical container, wherein the scented, shaped material includes a plastic composition, preferably a thermoplastic material, blended with a scent imparting material. In one embodiment, the scented, shaped material can be formed in the shape of a container for holding an adsorbing composition such as a desiccant or a gas adsorbing material. Alternatively, the scented material can be layered to form an inner portion of the pharmaceutical or neutraceutical container.

### CROSS-REFERENCE TO RELATED APPLICATIONS

This application claims priority from U.S. Provisional Application Ser. No. 60/720,138, filed on Sep. 23, 2005. This application is also a continuation-in-part application claiming priority from U.S. patent application Ser. No. 10/996,916, filed on Nov. 24, 2004, which is a continuation-in-part application claiming priority from U.S. patent application Ser. No. 10/328,579, filed on Dec. 24, 2002, which application claims priority from provisional application 60/375,841, filed on Apr. 25, 2002.

### BACKGROUND OF INVENTION

This invention relates to scented pharmaceutical and neutraceutical vessels, particularly pharmaceutical or neutraceutical vessels containing a scented shaped material or container placed within, secured within or forming a portion of the pharmaceutical or neutraceutical vessel.

Some neutraceutical or pharmaceutical products which are packaged in conventional containers, such as conventional glass or plastic bottles, have an unpleasant and/or unappetizing odor. An example is the odor produced by fish oil capsules. In current practice, the odor of these neutraceutical or pharmaceutical products may escape from the container, thereby creating an unpleasant environment.

In addition, some neutraceutical or pharmaceutical products degrade when they are exposed to moisture or certain gases, such as oxygen, for extended periods of time. Reduced levels of moisture and/or certain gases, such as oxygen, within the containers may be difficult to maintain once the container for the neutraceutical or pharmaceutical products has been opened. To address these problems, it has become standard practice to place a moisture absorbing material, such as a desiccant canister, and/or a gas absorber within the containers. One particular type of a gas absorber is an oxygen absorber.

The use of scented oils with selected plastic materials is known for certain limited applications. For example, U.S. Pat. No. 3,553,296 discloses a process for manufacturing a scented polyolefin that may have utility as an artificial flower, in the cosmetic industry or for the preparation of garbage bags. A method of providing scent to a product container by entrapping scented oil within a polymer matrix within a container, wherein the container is comprised of a material which is incompatible with the scented oil, is also disclosed in U.S. Pat. No. 4,540,721.

Notwithstanding these limited examples of uses of scented plastic materials, the concept of preparing a pharmaceutical or neutraceutical vessel containing a scented, shaped insert or container has not been disclosed. Further, incorporation of a scent imparting material as a component of a moisture or gas adsorbing container for use within neutraceutical or pharmaceutical containers has not been disclosed. Such products shows great utility for solving multiple problems that exist with conventional pharmaceutical and neutraceutical containers. In addition, utilization of a scented material to form only an inner layer of a pharmaceutical or neutraceutical container provides a surprising ability to decrease the impact of unwanted odors from products present in the containers. The addition of a color pigment to such a container can add further utility to the container.

These and other objects are obtained by the composition, process for the preparation of the composition and process of use of the composition in the neutraceutical and pharmaceutical industry.

### SUMMARY OF THE INVENTION

The invention includes a pharmaceutical or neutraceutical vessel containing a scented, shaped material, comprising a plastic composition blended with a scent imparting material, and optionally a color pigment and/or other additive, placed within the pharmaceutical or neutraceutical vessel.

The invention further includes a pharmaceutical or neutraceutical vessel containing a scented, shaped material, wherein the scented shaped material is a component of a moisture adsorbing container and/or a gas adsorbing container, particularly an oxygen absorbing container, which is placed within the pharmaceutical or neutraceutical container.

The invention further includes a pharmaceutical or neutraceutical vessel containing a scented shaped material, wherein the scented shaped material is formed, at least partially, of an adsorbent polymeric composition, wherein the adsorbent polymeric composition comprises a single thermoplastic material or a combination of thermoplastic materials, and at least one adsorbent, wherein the adsorbent is concentrated near the surface of the polymeric composition.

The invention further includes a pharmaceutical or neutraceutical vessel containing a scented shaped material, wherein the scented shaped material is formed, at least partially, of an absorbent polymeric composition, wherein the absorbent polymeric composition comprises only a single polymeric material and at least one moisture absorbent and/or a gas absorber, and wherein the quantity of the moisture absorbent within the polymeric material is substantial.

### DETAILED DESCRIPTION OF THE INVENTION

The invention comprises a pharmaceutical or neutraceutical vessel useful for holding drugs, vitamins, minerals or medical supplements with unpleasant odors containing a scented, shaped material, wherein the scented, shaped material is at least partially produced from a plastic composition blended with a scent imparting material, wherein the scented, shaped material is located within, is secured within or forms a portion of the pharmaceutical or neutraceutical vessel.

In one embodiment the pharmaceutical or neutraceutical vessel comprises a conventional container manufactured from conventional materials, such as plastic or glass. Placed within this pharmaceutical or neutraceutical container is the scented, shaped material. The scented, shaped material is preferably formed from a plastic material blended with the scent imparting material. Among the plastic materials that can be utilized are a single thermoplastic materials that is compatible with the pharmaceutical or neutraceutical products, which are placed within the container and/or which can be easily blended with the desired scent imparting material. Alternatively, the plastic materials can include more than one thermoplastic or thermoset material. The plastic materials may be selected from thermoplastic materials such as, but not limited to, polystyrenes, polyolefins, polyethylene, polypropylene, polyacrylates, polymethacrylates, polyamides, polyesters, and polyvinyl chloride. Non-limiting examples of copolymers include: styrene-butadiene rubbers (SBR), styrene-ethylene-butadiene-styrene copolymers (SEBS), butyl rubbers, ethylene-propylene rubbers (EPR), ethylene-propylene-diene monomer rubbers (EPDM), ethylene-vinyl acetate copolymers (EVA), ethylene-acrylate or butadiene-acrylonitrile, maleic anhydride modified polymers and copolymer, and grafted copolymers. In one preferred embodiment, where a single thermoplastic is utilized, the plastic material is polypropylene or polyethylene, preferably a high density polyethylene.

In another preferred embodiment, the neutraceutical or pharmaceutical container can be produced solely or partially from an adsorbent polymeric composition, which includes one or more thermoplastic materials and at least one adsorbent material for adsorbing moisture, gases, such as oxygen, and/or other chemical compounds, as described hereinafter. (For purposes of this invention the terms “absorbent” or “absorbing” and “adsorbent” or “adsorbing” have the same, all encompassing meaning.) Examples of acceptable adsorbent polymeric compositions are disclosed in U.S. patent application Ser. No. 10/996,916, filed on Nov. 24, 2004, which application is incorporated herein by reference. The thermoplastic material can be any material that exhibits thermoplastic properties, including but not limited to, a single thermoplastic material, such as polypropylene or polyethylene, a copolymer of two or more monomers, a mixture of two or more polymers from single monomers, a mixture of two or more copolymers and a mixture of at least one polymer from a single monomer and at least one copolymer. Non-limiting examples of polymers from single monomers include: polystyrenes, polyolefins, polyethylene, polypropylene, polyacrylates, polymethacrylates, polyamides, polyesters, and polyvinyl chloride. Non-limiting examples of copolymers include: styrene-butadiene rubbers (SBR), styrene-ethylene-butadiene-styrene copolymers (SEBS), butyl rubbers, ethylene-propylene rubbers (EPR), ethylene-propylene-diene monomer rubbers (EPDM), ethylene-vinyl acetate copolymers (EVA), ethylene-acrylate or butadiene-acrylonitrile, maleic anhydride modified polymers and copolymers, and grafted copolymers.

When blends of thermoplastic materials are used, it has been observed that one of the components of the thermoplastic blends tends to enrich at the surface together with the adsorbent material while the other component tends to enrich towards the center of the composition. However, it has been discovered that only in the melted state will the adsorbent material tend to migrate towards the surface of the composition. Care must be taken to prepare such articles so that the adsorbent materials and the thermoplastic material exhibit the separation described hereinafter while in the molten or flowable state. For example, the thermoplastic material may be prepared from a blend of linear low density polyethylene (LLDPE), low density polyethylene (LDPE) and ethylene vinyl acetate (EVA) copolymer, wherein each of the components includes an ethylene monomeric unit. “Separation” as used herein defines a concentration gradient difference and does not necessarily mean 100% separation of the components into distinct phases. Similarly, “layered” as used herein means a significant change in concentration gradient such that the product appears to be layered, and does not necessarily mean a layer of one component and a second layer of a different component. “Gradient” means that the concentration of any component of the absorbing polymeric material varies with distance from the surface of a product manufactured from the absorbing polymeric material.

In order to achieve this phase separation, it has been found preferable to use as a thermoplastic component a blend of at least one polymer derived from a single monomer with at least one copolymer. Preferably the copolymer contains the monomer of the single monomer component so that the two polymers are compatible. If two or more copolymers are mixed to form the thermoplastic material, they should preferably contain at least one common monomer. The adsorbent can be any material capable of adsorbing moisture, or otherwise removing moisture from a surrounding atmosphere, or any material capable of adsorbing or otherwise removing other chemical compounds, such as but not limited to gas compounds, such as, but not limited to, oxygen, carbon dioxide, carbon monoxide, ethylene and amine complexes, from the atmosphere. Herein, the term “adsorbent” includes but is not limited to the term, dehydrating agent, desiccant or absorbent. Non-limiting examples of adsorbents include silica gel, desiccant clay, molecular sieves, zeolites or combinations thereof.

The relative concentration of thermoplastic material to adsorbent may vary depending on the thermoplastic material and the absorbent used. In a preferred embodiment, the polymeric structure comprises from about 20 wt % to about 85 wt % thermoplastic material and from about 15 wt % to about 80 wt % adsorbent.

Where applicable, compositions of the adsorbent polymeric composition further include appropriate quantities, up to about 10 percent, of organic or inorganic additives that are useful in the field of plastic such as plasticizers, stabilizers, elastomers, dyes and pigments. The composition may be customized to include certain pigments and/or colorants. It is often desirable that the manufactured article have a particular color. A particular color may, for example, enhance aesthetic appeal of the article and may serve to identify the particular brand or manufacturer. Suitable pigments of black, white or colored pigments, as well as extenders may be used. Examples of useful pigments include, without limitation, titanium oxide, zinc oxide, zinc sulfide, barium sulfate, aluminum silicate, calcium silicate, carbon black, black iron oxide, copper chromite black, yellow iron oxides, red iron oxides, brown iron oxides, ocher, sienna, umber, hematite, limonite, mixed iron oxide, chromium oxide, Prussian blue, chrome green, chrome yellow, manganese violet and other well known pigments. Dyes may be employed instead of pigments or in addition to the pigments.

The absorbent polymeric material preferably does not include wicking fibers, as these fibers may burn or melt during the manufacturing process. The inclusion of fibers to act as a wick for moisture is unnecessary because of the increased moisture adsorbency of the layered structure of the composition.

Surprisingly, it has been found that products formed from the absorbent polymeric compositions exhibiting an accumulation of absorbing agent in a “migration zone” in a gradient towards the surface show distinct advantages in moisture adsorbency compared to structures that contain the same concentration of adsorbing agent throughout the product (monolithic structures) and structures that contain an adsorbing agent only at the surface.

The polymeric structure of the adsorbent polymeric composition is produced by forming and setting the thermoplastic material after it has been dosed with the adsorbent. The polymeric structure may be produced by common plastic manufacturing processes, such as extrusion, co-extrusion, injection molding, bi-injection molding, blow molding, and any other methods that involve melting the thermoplastic material to an essentially liquid state. For example, the polymeric structure may be produced by the steps of heating the selected thermoplastic material (or combination of materials) until the thermoplastic is viscous, adding the selected adsorbent, blending the adsorbent into the melted thermoplastic, extruding the thermoplastic—adsorbent blend, and cooling the thermoplastic—adsorbent blend. The polymeric structure can then be cut or ground or processed by other means known in the art. Preferably the blend should be produced using a low shear technique, i.e. less than about 100s⁻¹.

The composition of this embodiment is prepared such that the adsorbing agent tends to concentrate in a gradient within the migration zone near the surface of the polymeric composition. In a preferred embodiment, the concentration of the adsorbing agent at the surface creates distinct layers of the composition, which are identifiable, i.e., a surface layer that is enriched in the adsorbing agent and an interior layer that is depleted of that same adsorbing agent.

The surface layers (usually on both opposite surfaces of products like strips and tubes) of the product made from the adsorbent polymeric material generally form relatively well defined “migration zones”, to which the adsorbing agent “migrates.” Within this migration zone the maximum concentration of the adsorbing agent at a given volume unit is from 2 to 10 times, preferably 2 to 6 times, higher than its concentration in the interior or core layer of the product. The concentration of the adsorbing agent within the migration zone preferably exhibits a gradient towards the surface. The concentration of the adsorbing agent at any location within the product and the extent of the migration of the adsorbing agent may be determined by infra-red microanalysis.

It has also been surprisingly discovered that the accumulation of the adsorbing agent at a given volume unit within the migration zone is substantially greater than the accumulation at a given volume unit throughout the interior layer of the product. It is surprisingly found that the percentage of the adsorbing agent present in the migration zones of a product formed from the adsorbent polymeric material is at least about 2%, preferably at least about 4%, and most preferably at least about 6% of the overall amount of adsorbing agent present in the product, with maximum amount present being no more than about 70%, preferably no more than 50% and most preferably no more than about 40% of the overall amount of absorbing agent.

In practice it has been found that the extrusion method of manufacture of the adsorbent polymeric composition provides for more separation phenomena than does injection molding. While not wanting to be bound by any particular theory, this phenomena is probably because the extrusion process provides for more directed and constant flow of material in a single direction which results in the copolymer migrating toward the surface of the composition, taking along the adsorbent material with it. With injection molding, the composition flows in one directions but then comes into contact with the walls of the injection mold causing a back flow and partially remixing of the liquid composition. Also, injection molding of the walls of the injection mold tends to rapidly cool the outer layers of the injected thermoplastic thereby preventing strong migration of the adsorbent material to the outer layers.

By way of example, an useful pharmaceutical or neutraceutical vessel may be prepared by forming an exterior shell out of a substantially water impermeable thermoplastic material, such as polyethylene or polypropylene. A full or partial liner may be formed out of the scented polyolefinic shaped material. The liner may either be formed inside the vessel in a dual injection mode or formed separately from the vessel and later inserted. The preferred method for forming the scented shaped material is extrusion and therefore the preferred method of forming such a liner would be a separate extrusion of the liner and molding of the vessel with assembly of the two parts. Bi-injection molding is also a preferred method for the formation of this vessel.

The scent imparting material can be chosen from a large variety of aromatic or scenting materials. Generally the scent imparting materials should be oil-soluble, as oil-soluble scented substances generally dissolve in the polyolefinic material of the invention. Alternatively, scented resins may be used. Further, the substances preferably have GRAS status as recognized by the Flavoring Extract Manufactory Association. Any perfume essence, flavor or aromatic material may be incorporated into the polyolefinic material of the invention. Although not wanting to be limited, particularly suitable oils can be chosen depending upon the designated use of the vessel by the consumer, such as a lemon oil, scented oils from flowers, such as lilac, honeysuckle, rose or carnation or other such oils with GRAS status.

The quantity of the scenting material that can be used, can be varied depending upon the particular application. The quantity of the scenting material should be from about 0.1 wt % to 30 wt %, preferably 1 to 10 wt % by weight of the scented, shaped material.

Various manufacturing process can be utilized to produce the scented shape material. In one preferred embodiment, the plastic composition selected is heated to its melting temperature, wherein the scent imparting material is added in a closed container. The two materials are then mixed thoroughly. The temperature of the melt should be constantly controlled during the process. The mixture of the plastic material with the scent imparting material is then directed through a plurality of orifices where the mixture is then solidified in the form of small pellets or beads. These pellets or beads form the “master pellets” which may then be admixed and liquefied with additional scented or unscented polyolefinic material, preferably unscented polyolefinic material, to produce the final polyolefinic material. In one embodiment the ratio of the master pellets to the unscented polyolefinic material is approximately 1:5 to 1:1. By this process, relatively large quantities of the scent imparting material can be added to the polyolefinic material to form an intermediate material prior to final blending of that scented plastic material master pellets with a larger quantity of the unscented plastic material. The quantity of scented plastic material that is added to the unscented plastic material can be modified depending upon the needs of the consumer. Color pigment additives and/or other additives may also be incorporated into the scented plastic material during processing.

After the blending of the unscented plastic material with the scented plastic material, the melted plastic material can be formed into any shape or design as is useful, such as in the form of canisters, strips, beads or other such shaped materials which can be placed within a pharmaceutical or neutraceutical vessel. In one preferred embodiment, the scented plastic material is formed into the shape of a canister with openings therein, which can be used to hold, for example, desiccant materials or gas absorbing materials, such as oxygen absorbing materials. Alternatively, these canisters can be formed from an unscented plastic material and the previously discussed adsorbent polymeric composition and the shaped, scented polyolefinic material can be placed within the canisters to segregate the scented plastic material from the materials placed within the pharmaceutical or neutraceutical vessel. Openings are then provided in the canisters to permit the scent to permeate the vessel. Alternatively, the canisters can be formed partially or wholly from a material which is permeable to the scent contained in the scented plastic material. Conventional designs for such canisters can be utilized, such as those shown in U.S. Pat. No. 5,759,241, which is incorporated herein by reference.

In an alternative embodiment, the scented plastic material can be incorporated into an unscented plastic material using a bi-injection molding process with a conventional plastic material or with the previously discussed adsorbent polymeric composition. In this embodiment, the conventional plastic material forms the inner or outer layer of the container and the scented polyolefinic material forms one of the other layers of the container. For example, the pharmaceutical or neutraceutical container can be formed with an outer layer of a conventional unscented, plastic material, while the inner layer is formed from the scented, shaped material.

In another preferred embodiment, a canister or other container may be placed within the pharmaceutical or neutraceutical container, wherein the canister or other shaped material is formed by bi-injection molding with the scented polymeric material forming the outer layer of that container. By this process only a small quantity of the scented polyolefinic material need be used. In using bi-injection molding procedure, the bi-injection molded product is formed by conventional injection molding processes.

It will be apparent from the forgoing that while particular forms of the invention have been illustrated various modifications can be made without departing from the scope of the invention. Accordingly, the invention is not intended to be limited by the specification of the application.

We claim:

1. A pharmaceutical or neutraceutical vessel containing a scented material comprising
a pharmaceutical or neutraceutical container; and
a scented, shaped material comprising a plastic composition blended with a scent imparting material, wherein the scented, shaped material is present within the pharmaceutical or neutraceutical container.

2. The pharmaceutical or neutraceutical vessel of claim 1, wherein the pharmaceutical or neutraceutical container comprises a container for holding drugs, vitamins, minerals or medical supplements.

3. The pharmaceutical or neutraceutical vessel of claim 1, wherein the scented, shaped material comprises a container for holding an adsorbent material.

4. The pharmaceutical or neutraceutical vessel of claim 1, wherein the scented, shaped material comprises a canister.

5. The pharmaceutical or neutraceutical vessel of claim 1, wherein the scented, shaped material comprises an inner layer of the pharmaceutical or neutraceutical container.

6. The pharmaceutical or neutraceutical vessel of claim 1, wherein the plastic composition of the scented, shaped material comprises an adsorbent polymeric composition.

7. The pharmaceutical or neutraceutical vessel of claim 6, wherein the adsorbent polymeric composition comprises a single thermoplastic material and at least one adsorbent.

8. The pharmaceutical or neutraceutical vessel of claim 6, wherein the adsorbent polymeric composition of the scented shaped material comprises at least one thermoplastic material and at least one adsorbent, wherein said adsorbent is concentrated near the surface of the scented, shaped material.

9. The pharmaceutical or neutraceutical vessel of claim 6, wherein the adsorbent polymeric composition comprises from about 25 to about 75 wt. % thermoplastic material and from about 25 to about 75% adsorbent.

10. The pharmaceutical or neutraceutical vessel of claim 6, wherein the adsorbent polymeric composition comprises a mixture of components chosen from polymers, copolymers and monomers, wherein each component of said composition includes a common monomeric unit.

11. The pharmaceutical or neutraceutical vessel of claim 7, wherein the adsorbent comprises a desiccant selected from the group consisting of silica gel, desiccant clay, molecular sieves, zeolite and combinations thereof.

12. The pharmaceutical or neutraceutical vessel of claim 7, wherein the adsorbent polymeric composition comprising from about 20 to about 80 wt. % thermoplastic material and from about 20 to about 80% adsorbent.

13. The pharmaceutical or neutraceutical vessel of claim 8, wherein at least one thermoplastic material comprises a mixture of components chosen from polymers, copolymers and monomers, wherein each component of said composition includes a common monomeric unit.

14. The pharmaceutical or neutraceutical vessel of claim 8, wherein the adsorbent comprises a desiccant selected from the group consisting of silica gel, desiccant clay, molecular sieves, zeolite or combinations thereof.

15. The pharmaceutical or neutraceutical vessel of claim 1, wherein the scent imparting material comprises from about 0.1 to about 30 wt. % of the scented shaped material.

16. The pharmaceutical or neutraceutical vessel of claim 1, wherein the scent importing material comprises an oil soluble scented substance.

17. The pharmaceutical or neutraceutical vessel of claim 1 further comprising a color pigment additive.

18. A process for the production of a pharmaceutical or neutraceutical vessel containing a scented material comprising
heating a polyolefinic material to its melting temperature;
adding a scented material to the heated polyolefinic material;
mixing the two materials thoroughly;
forming pellets or beads from the mixed material;
reheating the pellets or beads to their melting temperature;
blending the melted pellets or beads with unscented polyolefinic material; and
preparing the scented shaped material.

19. The process of claim 18 wherein the ratio of the pellets and beads by weight to the unscented polyolefinic material is approximately 1:5 to 1:1.

20. The process of claim 18 further comprising mixing a color additive with the polyolefinic material.
